SEQUENCE LISTING (28 sequences)

SEQ ID NO: 1 - DNA, 20 bp, Artificial Sequence
  Description of Artificial Sequence Synthetic oligonucleotide
gatcaacaaatgtcatgagt

SEQ ID NO: 2 - DNA, 20 bp, Artificial Sequence
  Description of Artificial Sequence Synthetic oligonucleotide
tcaacaaatgtcatgagtgg

SEQ ID NO: 3 - DNA, 20 bp, Artificial Sequence
  Description of Artificial Sequence Synthetic oligonucleotide
acaaatgtcatgagtggctg

SEQ ID NO: 4 - DNA, 20 bp, Artificial Sequence
  Description of Artificial Sequence Synthetic oligonucleotide
gtcgcaacttacaaacgaag

SEQ ID NO: 5 - DNA, 20 bp, Artificial Sequence
  Description of Artificial Sequence Synthetic oligonucleotide
caacttacaaacgaagtata

SEQ ID NO: 6 - DNA, 21 bp, Artificial Sequence
  Description of Artificial Sequence Synthetic oligonucleotide
cttacaaacgaagtatagatc

SEQ ID NO: 7 - DNA, 20 bp, Artificial Sequence
  Description of Artificial Sequence Synthetic oligonucleotide
tacaaacgaagtatagatct

SEQ ID NO: 8 - RNA, 23 nt, Artificial Sequence
  Description of Artificial Sequence Synthetic oligonucleotide
aacauucaacgcugucggugagu

SEQ ID NO: 9 - RNA, 20 nt, Artificial Sequence
  Description of Artificial Sequence Synthetic oligonucleotide
caccgaccguugacuguacc

SEQ ID NO: 10 - DNA, 21 bp, Artificial Sequence
  Description of Artificial Sequence Synthetic oligonucleotide
acuuaaccggcauaccggctt

SEQ ID NO: 11 - DNA, 21 bp, Artificial Sequence
  Description of Artificial Sequence Synthetic oligonucleotide
gccgguaugccgguuaagutt

SEQ ID NO: 12 - DNA, 22 bp, Artificial Sequence
  Description of Artificial Sequence Synthetic oligonucleotide
catgtcatgtgtcacatctctt

SEQ ID NO: 13 - DNA, 21 bp, Artificial Sequence
  Description of Artificial Sequence Synthetic oligonucleotide
ctcaccgacagcgttgaatgt

SEQ ID NO: 14 - DNA, 23 bp, Artificial Sequence
  Description of Artificial Sequence Synthetic oligonucleotide
actcaccgacagcgttgaatgtt

SEQ ID NO: 15 - DNA, 21 bp, Artificial Sequence
  Description of Artificial Sequence Synthetic oligonucleotide
ctcaccgacagcgttgaatgt

SEQ ID NO: 16 - DNA, 16 bp, Artificial Sequence
  Description of Artificial Sequence Synthetic oligonucleotide
tagcttatcagactga

SEQ ID NO: 17 - DNA, 16 bp, Artificial Sequence
  Description of Artificial Sequence Synthetic oligonucleotide
tagcttatcagactga

SEQ ID NO: 18 - DNA, 18 bp, Artificial Sequence
  Description of Artificial Sequence Synthetic oligonucleotide
tagcttatcagactgatg

SEQ ID NO: 19 - DNA, 17 bp, Artificial Sequence
  Description of Artificial Sequence Synthetic oligonucleotide
aactagtgcgcagcttt

SEQ ID NO: 20 - DNA, 15 bp, Artificial Sequence
  Description of Artificial Sequence Synthetic oligonucleotide
aactagtgcgcagct

SEQ ID NO: 21 - DNA, 15 bp, Artificial Sequence
  Description of Artificial Sequence Synthetic oligonucleotide
aactagtgcgcagct

SEQ ID NO: 22 - DNA, 18 bp, Artificial Sequence
  Description of Artificial Sequence Synthetic oligonucleotide
tagcttatcagactgatg

SEQ ID NO: 23 - DNA, 17 bp, Artificial Sequence
  Description of Artificial Sequence Synthetic oligonucleotide
aactagtgcgcagcttt

SEQ ID NO: 24 - DNA, 51 bp, Artificial Sequence
  Description of Artificial Sequence Synthetic oligonucleotide
aatgcactagttcaacatcagtctgataagctagctcagcaagcttaatgc

SEQ ID NO: 25 - DNA, 51 bp, Artificial Sequence
  Description of Artificial Sequence Synthetic oligonucleotide
gcattaagcttgctgagctagcttatcagactgatgttgaactagtgcatt

SEQ ID NO: 26 - DNA, 51 bp, Artificial Sequence
  Description of Artificial Sequence Synthetic oligonucleotide
aatgcactagtcgccaatatttacgtgctgctagctcagcaagcttaatgc

SEQ ID NO: 27 - DNA, 50 bp, Artificial Sequence
  Description of Artificial Sequence Synthetic oligonucleotide
gcattaagcttgctgagctagcagcacgtaaatatggcgactagtgcatt

SEQ ID NO: 28 - DNA, 59 bp, Artificial Sequence
  Description of Artificial Sequence Synthetic oligonucleotide
cgactagttgtttacagtactcaccgacagcgttgaatgtttgcttcatatctagagtc

(19) United States
(12) Patent Application Publication
(10) Pub. No.: US 2010/0113284 A1
(43) Pub. Date: May 6, 2010

(54) SMALL INTERFERING RNA (SIRNA) TARGET SITE BLOCKING OLIGOS AND USES THEREOF

(76) Inventors: Alexander Aristarkhov, Chestnut Hill, MA (US); Søren Morgenthaler Echwald, Humlebaek (DK); Niels Montano Frandsen, Birkeroed (DK)

Correspondence Address: CLARK & ELBING LLP, 101 FEDERAL STREET, BOSTON, MA, 02110, US

(21) Appl. No.: 12/418,076

(22) Filed: Apr. 3, 2009

(30) Foreign Application Priority Data
Apr. 4, 2008 (DK) PA200800496

(51) Int. Cl. C40B 30/00; C07H 21/00; C12N 5/00; C12Q 1/68

(52) U.S. Cl. 506/7; 536/22.1; 435/375; 435/6

(57) ABSTRACT

The present invention relates to nucleic acids designed to prevent the binding of single strand RNA in protein complexes originatig from small interfering RNA (siRNA) or small hairpin RNA (shRNA) and uses thereof.

### CROSS-REFERENCE TO RELATED APPLICATIONS

This application claims the benefit of Danish Patent Application Number PA 2008 00496, filed Apr. 4, 2008.

The present invention relates to nucleic acids designed to prevent the binding of single strand RNA in protein complexes originatig from small interfering RNA (siRNA) or small hairpin RNA (shRNA) and uses thereof.

### BACKGROUND OF THE INVENTION

The present invention relates to the study and modulation of the effect of small RNAs on target nucleotide sequences in a wide variety of nucleic acid samples and more specifically to the methods employing the design and use of oligonucleotides that are useful for preventing the binding of siRNA, especially, to RNA target sequences, such as siRNA target sites.

### RNA Interference (RNAi)

Since Fire (Nature 391; 806-811, 1998) made the observation that RNA could be introduced into cells in C. elegans and block gene expression, and the subsequent discovery that this mechanism termed RNA interference (RNAi) is active in mammals (Elbashir et al., Nature 411; 494-498, 2001; McCaffrey et al., Nature 418; 38-39, 2002), RNAi has advanced into becoming a powerful genetic tool and promising biotherapeutic for a wide array of diseases.

The RNAi response is triggered by the presence of double-stranded RNA (dsRNA; over 100 nt) in cells. The dsRNA is degraded into short doublestranded fragments (approximately 21-23 nt long) known as small interfering RNA (siRNA) by an RNAse III-type enzyme, Dicer. The generated siRNA enters the RNA-induced silencing complex (RISC), which becomes activated upon guide (antisense) strand selection (Maritnez et al., Cell 110; 563-574, 2002). Guide strand selection is based on the relative thermodynamic stabilities of the two duplex ends and it is the least stable 5′ end of the duplex that is recognized and asymmetrically unwound by the Piwi-Argonaute-Zwille (PAZ) domain of argonaute 2, a multifunctional protein within the RISC. The incorporated strand acts as a guide for the activated RISC complex to selectively degrade the complementary mRNA and prevent translation. The argonaute 2 protein is responsible for mRNA cleavage via its PIWI domain, which adopts an RNase H-like structure (Martin and Caplen, Rev. Genomics Hum. Genet. 8; 81-108, 2007; Parker and Barford, Trends Biochem. Sci. 31; 622-630, 2006).

Small Interfering RNAs (siRNAs)

One of the perceived advantages using siRNA as a functional genomics tool is its ability to silence genes in a sequence-specific manner. While long double-stranded RNA molecules can be employed to induce RNAi in lower eukaryotes, siRNAs have to be used for gene silencing in mammalian cells in order to prevent the activation of an unspecific interferon response (Elbashir et al., Nature; 411; 494-498, 2001). Gene expression in cell cultures can be conveniently blocked by either transfecting the siRNA into cells (Janowski et al.; Nat. Protoc. 1;436-443, 2006) or by introducing a vector that can express the siRNA within the cells (Tiscornia et al., Nat. Protoc; 1; 234-240, 2006). A 7 nt complementation between the siRNA and the target site has been found to be sufficient to cause gene silencing and sequences surrounding the siRNA target sites are also important for the silencing effect (Lin et al., Nucleic Acids Res 33(14); 4527-4535, 2005).

For the RNAi pathway to be a useful tool in the research and therapeutic venues, the siRNA, must be designed to be both potent and specific in its targeting of messenger RNA transcripts. Multiple design algorithms have been developed that enhance the selection of highly functional duplexes and the accurate prediction of siRNA target gene knockdown (Naito et al., Nucleic Acid Res. 32; W124-W129, 2004; Jagla et al., RNA 11; 864-872, 2005; Huesken et al. Nat. Biotechnol. 23; 995-1001, 2005). However, less is known about the parameters that contribute to siRNA specificity. Unintended gene modulation can result from lipid delivery reagents and siRNA induction of the innate cellular immunity. A third contributor to unintended gene knockdown is associated with off-targeting.

### Off-Targeting

Off target gene silencing is an RNAi-mediated event that results in changes in the expression of several genes by different mechanisms including global up/down-regulation of genes using high concentrations of siRNA, the induction of an interferon response, miRNA-like translational inhibition and mRNA degradation mediated by partial sequence complementarity. Recent work has indicated that some off-target effects are caused by the siRNAs cooperating with endogenous miRNAs at optimally spaced target sites to down-regulate mRNAs (Saetrom et al., Nucleic Acids Res. 35(7); 2333-2342, 2007). Off-target effects can be mediated by either strand of the siRNA and have been documented to occur when 15 base pairs, and as few as 11 contiguous base pairs, of sequence identity exist between the siRNA and off-target transcript (Jackson et al., Nat. Biotechnol. 21; 635-637; 2003). As described in (Jackson et al., Nat. Biotechnol. 21; 635-637; 2003) 8 different siRNAs designed to target the MAPK14 gene revealed few genes regulated in common by different siRNAs to the same target gene when transfected into Hela cells.

A problem in evaluating off-target effects of siRNAs is to differentiate between direct effects of the siRNA on targets and effects which are secondary to the effect on the primary target. In (Jackson et al., Nat. Biotechnol. 21; 635-637; 2003), the authors concluded that since certain transcripts were regulated by the siRNA transfection earlier than the target gene protein (14 hours versus 40 hours post transfection), these effects were likely to be direct off-target effects from the siRNA rather than secondary effects following the regulation of the protein target.

As off-targeting can induce measurable phenotypes, including potential toxicity, and problems in data interpretation (Lin et al., Nucleic Acids Res. 33; 4527-4535, 2005), it represents one of the largest impediments for therapeutic and phenotypic screening applications for RNAi.

Comparison of validated off-target data set with in silico predicted off-targets recently showed that overall identity, except for near-perfect matches, does not accurately predict off-targeted genes (Birmingham et al., Nature Methods 3(3); 199-204, 2006). Perfect matches between the hexamer or heptamer seed region (positions 2-7 or 2-8 of the antisense strand) of an siRNA and the 3′ UTR were found to be associated with off-targeting. Nevertheless, only a small percentage of transcripts that contain seed sites are significantly down-regulated by the siRNAs. These results indicate a strong mechanistic parallel between siRNA off-targeting and microRNA-mediated gene regulation and reveal that current protocols used to minimize off-target effects (f.ex. blastn and Smith-Waterman algorithm) have little merit aside from eliminating the most obvious off-targets.

Microarray-based gene expression analysis has been used as a method of off-target identification (Jackson et al. Nat. Biotechnol. 21; 635-637; 2003, PCT Patent Application No. WO 2005/18534).

In conclusion, a challenge in functional analysis of siRNA and the exploitation of RNA interference as a gene knockdown tool for research and in therapy is the ability of siRNAs to target multiple target nucleotides in an undesired manner. The present invention provides the design and development of novel oligonucleotide compositions and sequences, providing an accurate, specific, and highly sensitive solution to specifically block a particular siRNA target site in a particular target nucleic acid without inducing degradation of the same target nucleic acid useful for determining the level of siRNA off-targeting.

### SUMMARY OF THE INVENTION

The present invention solves the current problems faced by conventional approaches used in studying and modulating the interaction of siRNAs with their target nucleic acid(s) (e.g., mRNAs) by providing a method for the design, synthesis, and use of novel oligonucleotide compositions with improved sensitivity and high sequence specificity for RNA target sequences. Such oligonucleotides include a recognition sequence at least partially complementary to the siRNA target site, wherein the recognition sequence may be substituted with high-affinity nucleotide analogues, e.g., LNA, to increase the sensitivity and specificity relative to conventional oligonucleotides, such as DNA oligonucleotides, for hybridization to short target sequences, e.g., siRNAs and siRNA target sites.

Accordingly, in one aspect the invention provides a nucleic acid binding to a region including a portion of a naturally occurring siRNA target site and, optionally, to a naturally occurring nucleic acid sequence adjacent to the siRNA target site. Preferably, the nucleic acids of the invention include a high affinity nucleic acid analog, e.g., LNA. The nucleic acid binds, for example, to the 3′ end or 5′ end of the siRNA target site. Alternatively, the nucleic acid binds to 100% of the siRNA target site. In another embodiment, at least 10%, e.g., at least 15, 20, 25, 30, 35, 40, 45, 50, 55, 60, 65, 70, 75, 80, 85, 90, or 95%, of the nucleic acid is not complementary to the siRNA target site. The nucleic acid is, for example, from 5-30 nucleotides, e.g., at least 10, 15, 20, or 25. In certain embodiments, the nucleic acid includes a plurality of high affinity nucleotide analogs, e.g., of the same or different type. For example, the nucleic acid may include up to 80%, e.g., up to 75, 70, 65, 60, 55, 50, 45, 40, 35, 30, 25, or 20%, of the high affinity nucleic acid analog or the high affinity nucleic acid analog, e.g., LNA, in combination with one or more additional analogs, e.g., 2′ OMe. Preferably, the plurality of analogs are disposed so that no more than four naturally occurring nucleotides occur consecutively.

The nucleic acid in a preferred embodiment is complementary to at least two nucleotides of the siRNA target site and at least three nucleotides in the naturally occurring nucleic acid sequence adjacent to the siRNA target site. The nucleic acid may be complementary to 2-6 nucleotides of the siRNA target site to which the seed sequence of the siRNA binds. A high affinity nucleic analog may or may not be disposed at the 3′ or 5′ end of the nucleic acid. The nucleic acid is also preferably RNase resistant. Preferably, the nucleic acid does not prevent production of the siRNA from its corresponding precursor dsRNA or shRNA. In other embodiments, the analogs are not disposed in regions capable of forming auto-dimers or intramolecular complexes.

The binding of the nucleic acid to the region desirably reduces the binding of the siRNA to the region, e.g., by at least 50%, 55%, 60%, 65%, 70%, 75%, 80%, 85%, or 90%. Alternatively, the nucleic acid binds to the region with a lower Kd than the siRNA in vivo. The nucleic acid may also have an increase in binding affinity to the region as determined by an increase in Tm of at least 2° C., compared to the naturally occurring RNA complement of the region.

In various embodiments, a nucleic acid of the invention specifically includes one or more of 2′-O-methyl-modified nucleic acids (2′-OMe), 2′-O-(2-methoxyethyl)-modified nucleic acids (2′-MOE), 2′-Deoxy-2′-fluoro-β-D-arabinoic acid (FANA), Cyclohexene nucleic acids (CeNA), Hexitol nucleic acids (HNA) and analogs thereof, Intercalating Nucleic Acids (INA), 2′-O,4′-C-Ethylene-bridged-Nucleic Acids (ENA), and peptide nucleic acid (PNA). In other embodiments, a nucleic acid of the invention does not include 2′-O-methyl-modified nucleic acids (2′-OMe); a nucleic acid of the invention does not include 2′-O-(2-methoxyethyl)-modified nucleic acids (2′-MOE); a nucleic acid of the invention does not include 2′-Deoxy-2′-fluoro-β-D-arabinoic acid (FANA); a nucleic acid of the invention does not include Cyclohexene nucleic acids (CeNA); a nucleic acid of the invention does not include Hexitol nucleic acids (HNA) or analogs thereof; a nucleic acid of the invention does not include Intercalating Nucleic Acids (INA); a nucleic acid of the invention does not include 2′-O,4′-C-Ethylene-bridged-Nucleic Acids (ENA); and/or a nucleic acid of the invention does not include peptide nucleic acids (PNA).

The invention further features a method of inhibiting the binding of a siRNA to a target site by contacting one or more nucleic acids of the invention with a cell expressing the target site. The contacting may occur in vitro or in vivo.

In another aspect, the invention features a method of identifying the presence of a siRNA target site by contacting a nucleic acid sample from a subject with one or more nucleic acids of the invention and determining whether the one or more nucleic acid binds to the sample.

In a further aspect, the invention features a method of verifying the presence of a siRNA target site by contacting a nucleic acid sample from a subject with one or more nucleic acids of the invention and determining an expression level of a nucleic acid comprising said target site or its translation product, wherein a change in the expression level of the nucleic acid comprising the target site or its translation product verifies the presence of the siRNA target site.

Furthermore, the invention features a method of verifying the presence of a siRNA target site, said method comprising predicting, such as by using a siRNA design algorithm, the presence of a siRNA target site in a nucleic acid, and contacting the nucleic acid sample with one or more nucleic acids of the invention and determining an expression level of a nucleic acid comprising the target site or its translation product, wherein a change in the expression level of a nucleic acid comprising the target site or its translation product verifies the presence of the siRNA target site.

The invention also features a methods of determining an off-target effect and whether binding of a siRNA to a siRNA target site is associated with any unintended effects, such as off-target effects, immune response activation and/or non-specific gene silencing.

In one embodiment a method of determining an off-target effect induced by a siRNA on an eukaryotic cell expressing a target site for the siRNA, comprises determining a phenotype of the eukaryotic cell after subjecting the cell to the siRNA and one or more nucleic acid(s) of the invention, binding to a region comprising a portion of the target site. Preferably, the determining the phenotype comprises determining the expression levels by array analysis as described herein of a plurality of different genes, such as at least 5 different genes, such as at least 10 different genes, such as at least 100 different genes, such as at least 1000 different genes, or such as at least 10,000 different genes, such as at least 25,000 different genes, and/or their translation products.

In a preferred embodiment the method of determining whether a phenotype induced by a siRNA in an eukaryotic cell expressing a target site for the siRNA is associated with any unintended effects, such as off-target effects, immune response activation and/or non-specific gene silencing, comprises determining a phenotype the eukaryotic cell, introducing the siRNA, and one or more nucleic acid(s) according to the present invention directed to the siRNA target site into the eukaryotic cell, determining a phenotype of the eukaryotic cell after introduction of the siRNA and one or more nucleic acid(s) according to the present invention, and comparing the two phenotypes, wherein if the phenotypes differ, the phenotype induced by the siRNA is associated with an off-target effect. In one embodiment the method of determining whether a phenotype induced by a siRNA is associated with any unintended effects, such as off-target effects, immune response activation and/or non-specific gene silencing, further comprises determining a phenotype of the eukaryotic cell after introduction of the siRNA but prior to introduction of the one or more nucleic acid(s) according to the present invention.

In preferred embodiments the determining the phenotypes in the cell populations comprises determining the expression levels by array analysis as described herein of a plurality of different genes and/or their translation products The plurality of different genes may comprise at least 5 different genes, such as 10 different genes, such as 100 different genes, such as 1000 different genes, such as 10,000 different genes, or such as 25,000 different genes.

The nucleic acids of the invention are not splice-splice switching oligomers, e.g., of the TNFR superfamily (U.S. 2007/0105807).

In another aspect, the invention provides a nucleic acid as described above with the expection that the region does not include a naturally occurring nucleic acid sequence adjacent to the siRNA target site. Such nucleic acids may be used in any of the methods described herein and have any of the features of the other nucleic acids of the invention, unless otherwise noted.

### BRIEF DESCRIPTION OF THE DRAWINGS

FIG. 1 illustrates examples of siRNA target site blocking oligos. (A) The siRNA target site blocking oligo recognize the entire sequence of a selected siRNA target site. (B) The siRNA target site blocking oligo recognizes a sequence comprising a portion of a selected siRNA target site and a gene-specific sequence adjacent to the siRNA target site.

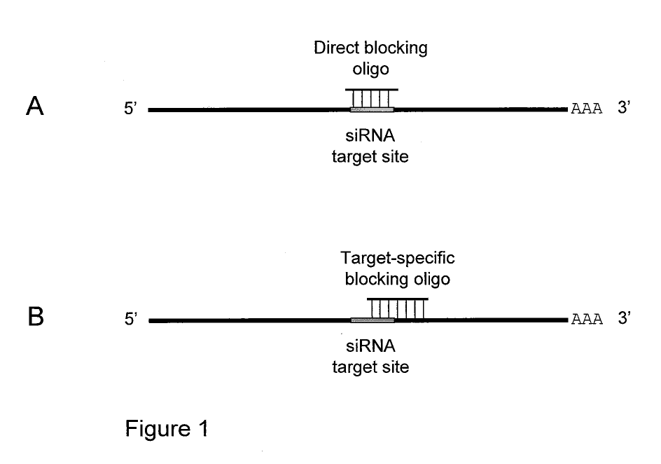

FIG. 2: Target site blocking pMIR-21 luciferase vector. The diagram shows the normalised expression level of the pMIR-21 reporter vector as a function of oligonucleotide concentration. The left panel shows results obtained from HeLa cells and the right panel results obtained from MCF7 cells.

FIG. 3: Target site blocking pMIR-16 “control” luciferase vector. The diagram shows the normalised expression level of the pMIR-16 reporter vector as a function of oligonucleotide concentration. The left panel shows results obtained from HeLa cells and the right panel results obtained from MCF7 cells.

FIG. 4: Localisation of target site blocker probes relative the mir181a target site. Underlined sequence: mir-181a perfect match target site; Italics indicate adjacent sequence from the reporter construct. TSB 1-7: sequence and alignment to reporter construct of target site blocking oligos. Tm: Predicted Tm's of target site blocker probes; SAS: self annealing score; TSB-1-7: Target site blocker probes 1-7. In the probe sequence, LNA nucleotides are denominated by capital letters. TSB-1 to 7 are synthesized using a phosphorothiorate backbone.

FIG. 5: First column (seule): cell line stably transfected with a vector containing a luciferase reporter construct with a mir-181a target site in the 3′UTR. “mir181+LNA controle”: Co-transfection of mir-181 mimic (RNA duplex see table 3) and 4 different concentrations of a LNA control oligo with no function (table 3). Mir-181 mimic effectively downregulates luciferase expression. “mir181+TSB1 exiqon”-“mir181+TSB7 exiqon”: Co-tansfection of mir-181 mimic (RNA duplex see table 3) and 4 different concentrations of a LNA-containing target site blocking oligo (TSB-1 to TSB-7, table 2). TSB-1, TSB-2 and TSB-5 effectively block the effect of the mir-181 mimic. “siRNA+LNA controle”: Co-transfection an siRNA with no effect on the mir-181 reporter construct (“controle mimic” RNA duplex see table 3) and 4 different concentrations of a LNA control oligo with no function (“LNA control”, table 3). Experiments performed in duplicates.

FIG. 6: First column (seule): cell line stably transfected with a vector containing a luciferase reporter construct with a mir-181a target site in the 3′UTR. “mir181+LNA contrl”: Co-transfection of mir-181 mimic (RNA duplex see table 3) and 5 different concentrations of a LNA control oligo with no function (table 3). Mir-181 mimic downregulates luciferase expression. “mir181+TSB1” - “mir181+TSB5”: Co-transfection of mir-181 mimic (RNA duplex see table 3) and 5 different concentrations of a LNA-containing target site blocking oligo (TSB-1 to TSB-7, table 2). TSB-1, TSB-2 and TSB-5 effectively block the effect of the mir-181 mimic. “siRNA+LNA controle”: Co-transfection of 5 different concentrations of an siRNA with no effect on mir-181 construct (“controle mimic” RNA duplex see table 3) and a LNA control oligo with no function (“LNA control”, table 3). “mir181+LNA ND (a highly potent mir-181 antisense oligo—positive control)”: Co-tansfection of mir-181 mimic (RNA duplex see table 3) and 5 different concentrations of a LNA-containing microRNA 181-antisense oligo (“LNA ND”, table 3). The mir-specific LNA ND oligo effectively blocks the effect of the mir-181 mimic. “mir181+LNA oligoold” and “mir181+LNA exiqon”: Co-tansfection of mir-181 mimic (RNA duplex see table 3) and 5 different concentrations of two different LNA-containing microRNA antisense oligos (“LNA oligoold” and “LNA exiqon”, table 3). The LNA knockdown oligos have no influence on the effect of the mir-181 mimic. Experiments performed in triplicates.

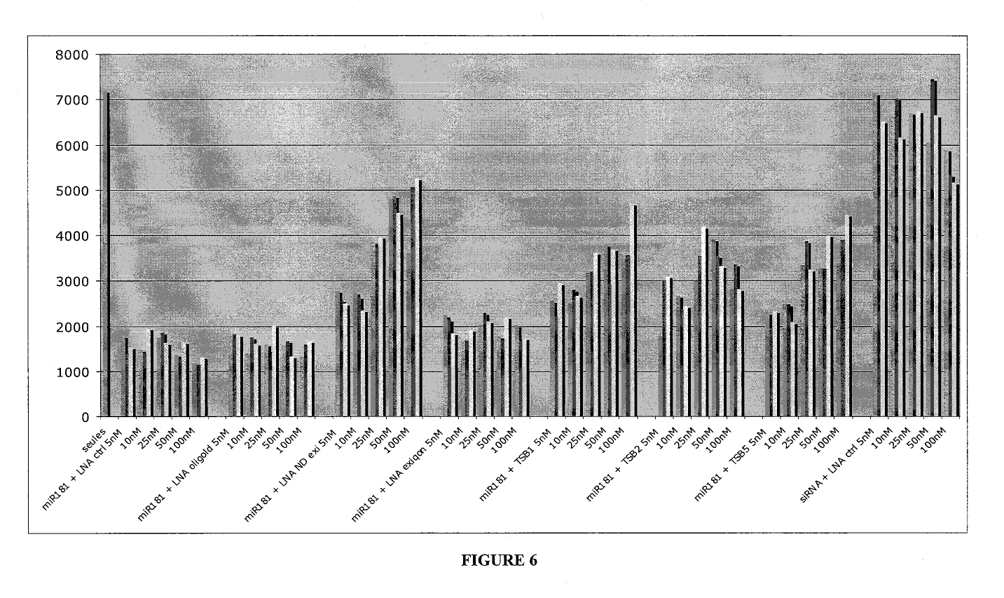

TABLE 2

| | Predicted | Self | | |
|---|---|---|---|---|
| | hybridization | hybridization | Probe | |
| Sequence | temperature | score | name | Backbone |
| gAtCaaCaAatGtCatGaGt | Tm = 70° C. | SAS = 42 | TSB-7 | Phosphorothioate |
| (SEQ ID NO: 1) | | | | |
| tCaAcaAatGTcatGaGtGg | Tm = 72° C. | SAS = 43 | TSB-6 | Phosphorothioate |
| (SEQ ID NO: 2) | | | | |
| aCaAatGTcatGaGtGGctG | Tm = 75° C. | SAS = 47 | TSB-5 | Phosphorothioate |
| (SEQ ID NO: 3) | | | | |
| gTcgCaaCtTaCaAacGaaG | Tm = 75° C. | SAS = 40 | TSB-1 | Phosphorothioate |
| (SEQ ID NO: 4) | | | | |
| cAaCtTaCaAaCGaaGtAtA | Tm = 70° C. | SAS = 42 | TSB-2 | Phosphorothioate |
| (SEQ ID NO: 5) | | | | |
| cTtaCaAaCGaaGtAtaGatC | Tm = 69° C. | SAS = 36 | TSB-3 | Phosphorothioate |
| (SEQ ID NO: 6) | | | | |
| tACaAaCGaaGtAtaGaTcT | Tm = 72° C. | SAS = 40 | TSB-4 | Phosphorothioate |
| (SEQ ID NO: 7) | | | | |

Tm: Predicted Tm's of target site blocker probes; SAS: self annealing score; TSB-1-7: Target site blocker probes 1-7. In the probe sequence, LNA nucleotides are denominated by capital letters.

TABLE 3

| | | |
|---|---|---|
| oligo Name | RNA oligo sequences | Backbone |

| | | |
|---|---|---|
| mir-1818a sense | aacauucaacgcugucggugagu | RNA |
| | (SEQ ID NO: 8) | |
| mir-181a anti-sense | caccgaccguugacuguacc | RNA |
| | (SEQ ID NO: 9) | |
| Control mimick sense | acuuaaccggcauaccggcdTdT | RNA |
| | (SEQ ID NO: 10) | |
| Control mimick anti- | gccgguaugccgguuaagudTdT | RNA |
| sense | (SEQ ID NO: 11) | |
| LNA control | catgtcaTGTGTCACatctctt | PO |
| | (SEQ ID NO: 12) | |
| LNA ND | cTcAccgAcaGcgTtgAaTgt | Phosphorothioate |
| | (SEQ ID NO: 13) | |
| “LNA oligoold” | actcaccgACAGCGTTgaatgtt | PO |
| | (SEQ ID NO: 14) | |
| “LNA Exiqon” | cTcAccgAcaGcgTtgAaTgt | PO |
| | (SEQ ID NO: 15) | |

In the probe sequence, LNA nucleotides are denominated by capital letters.

### DEFINITIONS

For the purposes of the subsequent detailed description of the invention the following definitions are provided for specific terms, which are used in the disclosure of the present invention:

The term “siRNA” refers to 19 to 25 nt-long double-stranded small interfering RNAs. They are processed from longer double-stranded RNAs (dsRNA) or small hairpin RNAs (shRNA) by the enzyme Dicer. siRNAs assemble in RISC-complexes wherein the incorporated strand acts as a guide to selectively degrade the complementary mRNA.

In the present context, the terms “blocking oligo” or “blocking molecule” refer to an oligonucleotide, which comprises a recognition sequence partly complementary to the target site of a siRNA.

“miRNA target site” or “microRNA target site” refers to a specific target binding sequence of a microRNA in a mRNA target. Complementarity between the miRNA and its target site need not be perfect.

Likewise, “siRNA target site” refers to a specific target binding sequence of a siRNA in a mRNA target. Complementarity between the siRNA and its target site need not be perfect.

The terms “seed region” or “seed sequence” refer to the 5′ end of a microRNA that is implicated in gene regulation by inhibition of translation and/or mRNA degradation or the portion of the guide strand in a siRNA that is analogous to the seed region of a microRNA.

In the present context, the term “expression level” when refering to a nucleic acid or a translation product refers to the steady-state amount of the nucleic acid or translation product present as determined by methods known in the art and described herein.

In the present context, the term “phenotype” refers to any observed quality of a cell or organism, such as its morphology, development, or behaviour, its transcriptional or translational state, or other aspects of the biological state.

The terms “off-target effect” or “off-targeting” in the present context refer to any gene silencing effect caused by siRNAs in non-target mRNAs through the RNAi mechanism.

In the present context “ligand” means something that binds. Ligands include biotin and functional groups such as: aromatic groups (such as benzene, pyridine, naphtalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-β-alanine, polyglycine, polylysine, peptides, oligo/polysaccharides, oligo/polyphosphates, toxins, antibiotics, cell poisons, and steroids, and also “affinity ligands”, i.e., functional groups or biomolecules that have a specific affinity for sites on particular proteins, antibodies, poly- and oligosaccharides, and other biomolecules.

The singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

“Transcriptome” refers to the complete collection of transcriptional units of the genome of any species. In addition to protein-coding mRNAs, it also represents non-coding RNAs, such as microRNAs, which have important structural and regulatory roles in the cell.

“Sample” refers to a sample of cells, or tissue or fluid isolated from an organism or organisms, including but not limited to, for example, skin, plasma, serum, spinal fluid, lymph fluid, synovial fluid, urine, tears, blood cells, organs, tumours, and also to samples of in vitro cell culture constituents (including but not limited to conditioned medium resulting from the growth of cells in cell culture medium, recombinant cells and cell components).

An “organism” refers to an entity alive at some time, including but not limited to, for example, human, mouse, rat, Drosophila, C. elegans, yeast, Arabidopsis thaliana, maize, rice, zebra fish, primates, domestic animals, etc.

The terms “detection probe” or “detection probe sequence” refer to an oligonucleotide including a recognition sequence complementary to a RNA target sequence, wherein the recognition sequence is substituted with a high-affinity nucleotide analogs, e.g., LNA, to increase the sensitivity and specificity compared to conventional oligonucleotides, such as DNA oligonucleotides, for hybridization to short target sequences, e.g., mature miRNAs, siRNAsas well as miRNA/siRNA binding sites in their cognate mRNA targets.

The terms “miRNA” and “microRNA” refer to 21-25 nt non-coding RNAs derived from endogenous genes and in the present context comprise the socalled mirtrons, produced from splicing of a short intron with hairpin potential (Berezikov et al., Mol. Cell 28; 328-336, 2007). The miRNAs are processed from longer (ca. 75 nt) hairpin-like precursors termed pre-miRNAs. MicroRNAs assemble in complexes termed miRNPs and recognize their targets by antisense complementarity. If the microRNAs match 100% their target, i.e., the complementarity is complete, the target mRNA is cleaved, and the miRNA acts like a siRNA. If the match is incomplete, i.e., the complementarity is partial, then the translation of the target mRNA is blocked.

The term “recognition sequence” refers to a nucleotide sequence that is complementary to a region within the target nucleotide sequence essential for sequence-specific hybridization between the target nucleotide sequence and the recognition sequence.

The term “label” as used herein refers to any atom or molecule which can be used to provide a detectable (preferably quantifiable) signal, and which can be attached to a nucleic acid or protein. Labels may provide signals detectable by fluorescence, radioactivity, colorimetric, X-ray diffraction or absorption, magnetism, enzymatic activity, and the like.

As used herein, the terms “nucleic acid”, “polynucleotide” and “oligonucleotide” refer to primers, probes, oligomer fragments to be detected, oligomer controls and unlabelled blocking oligomers and shall be generic to polydeoxyribonucleotides (containing 2-deoxy-D-ribose), to polyribonucleotides (containing D-ribose), and to any other type of polynucleotide which is an N glycoside of a nucleobase, e.g., purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA. The oligonucleotide is comprised of a sequence of approximately at least 3 nucleotides, preferably at least about 6 nucleotides, and more preferably at least about 8-30 nucleotides corresponding to a region of the designated target nucleotide sequence. “Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

The terms “oligonucleotide” or “nucleic acid” intend a polynucleotide of genomic DNA or RNA, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature; and/or (3) is not found in nature. Because mononucleotides are reacted to make oligonucleotides in a manner such that the 5′-phosphate of one mononucleotide pentose ring is attached to the 3′ oxygen of its neighbour in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have a 5′ and 3′ ends. When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide.

The linkage between two successive monomers in a nucleic acid consists of 2 to 4, desirably 3, groups/atoms selected from —CH₂—, —O—, —S—, —NR^{H}—, >C═, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂, —O—NR^{H}—, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{H})—O—, where R^{H} is selected form hydrogen and C₁₋₄-alkyl, and R″ is selected from C₁₋₆-alkyl and phenyl, are especially desirable. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring as substituent P* at the 3′-position, whereas the right-hand side is bound to the 5′-position of a preceding monomer.

By the term “SBC nucleobases” is meant “Selective Binding Complementary” nucleobases, i.e., modified nucleobases that can make stable hydrogen bonds to their complementary nucleobases, but are unable to make stable hydrogen bonds to other SBC nucleobases. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called ^{2S}U)(2-thio-4-oxo-pyrimidine) and 2-thio-thymine (T′, also called ^{2S}T)(2-thio-4-oxo-5-methyl-pyrimidine). The pairs A-^{2S}T and D-T have 2 or more than 2 hydrogen bonds whereas the D-^{2S}T pair forms a single (unstable) hydrogen bond. Likewise SBC nucleobases include pyrrolo-[2,3-d]pyrimidine-2(3H)-one (C′, also called PyrroloPyr) and hypoxanthine (G′, also called I)(6-oxo-purine), where the pairs PyrroloPyr-G and C-I have 2 hydrogen bonds each whereas the PyrroloPyr-I pair forms a single hydrogen bond.

“SBC LNA oligomer” refers to a “LNA oligomer” containing at least one LNA monomer where the nucleobase is a “SBC nucleobase”. Generally speaking SBC LNA oligomers include oligomers that besides the SBC LNA monomer(s) contain other modified or naturally occurring nucleotides or nucleosides. By “SBC monomer” is meant a non-LNA monomer with a SBC nucleobase. By “isosequential oligonucleotide” is meant an oligonucleotide with the same sequence in a Watson-Crick sense as the corresponding modified oligonucleotide e.g. the sequences agTtcATg is equal to agTscD^{2S}Ug where s is equal to the SBC DNA monomer 2-thio-t or 2-thio-u, D is equal to the SBC LNA monomer LNA-D, and ^{2S}U is equal to the SBC LNA monomer LNA ^{2S}U.

The complement of a nucleic acid sequence as used herein refers to an oligonucleotide which, when aligned with the nucleic acid sequence such that the 5′ end of one sequence is paired with the 3′ end of the other, is in “antiparallel association.” Bases not commonly found in natural nucleic acids that may be included in the nucleic acids of the present invention include, for example, inosine and 7-deazaguanine. Complementarity may not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the duplexes have disassociated. Stability can also be used as a measure of binding affinity of an oligonucleotide towards its target.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acid Research, 25: 4429-4443, 1997. The term “nucleobase” thus includes not only the known purine and pyrimidine heterocycles, but also heterocyclic analogues and tautomers thereof. Further naturally and non naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30: 613-722, 1991 (see, especially pages 622 and 623, and in the Concise Encyclopedia of Polymer Science and Engineering, J. I. Kroschwitz Ed., John Wiley & Sons, pages 858-859, 1990, Cook, Anti-Cancer Drug Design 6: 585-607, 1991, each of which are hereby incorporated by reference in their entirety).

The term “nucleobase” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole or a 5-nitroindole. Other preferred compounds include pyrene and pyridyloxazole derivatives, pyrenyl, pyrenylmethylglycerol derivatives and the like. Other preferred universal bases include, pyrrole, diazole or triazole derivatives, including those universal bases known in the art.

By “LNA” or “LNA monomer” (e.g., an LNA nucleoside or LNA nucleotide) is meant a nucleoside or nucleotide analogue that includes at least one LNA monomer. LNA monomers as disclosed in PCT Publication WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA monomers and their method of synthesis also are disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12(1):73-76, 2002; Hakansson et al., Bioorg. Med. Chem. Lett. 11(7):935-938, 2001; Koshkin et al., J. Org. Chem. 66(25):8504-8512, 2001; Kvaerno et. al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998.

Preferred LNA monomers, also referred to as “oxy-LNA” are LNA monomers which include bicyclic compounds as disclosed in PCT Publication WO 03/020739 wherein the bridge between R⁴′ and R²′ as shown in formula (I) below together designate —CH₂—O— or —CH₂—CH₂—O—.

By “LNA modified oligonucleotide” or “LNA substituted oligonucleotide” is meant an oligonucleotide comprising at least one LNA monomer of formula (I), described infra, having the below described illustrative examples of modifications:

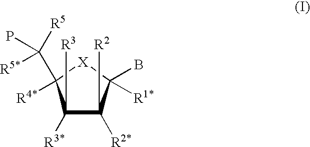

wherein X is selected from —O—, —S—, —N(R^{N})—, —C(R⁶R^{6*})—, —O—C(R⁷R^{7*})—, —C(R⁶R^{6*})—O—, —S—C(R⁷R^{7*})—, —C(R⁶R^{6*})—S—, —N(R^{N*})—C(R⁷R^{7*})—, —C(R⁶R^{6*})—N(R^{N*})—, and —C(R⁶R^{6*})—C(R⁷R^{7*}).

B is selected from a modified base as discussed above e.g. an optionally substituted carbocyclic aryl such as optionally substituted pyrene or optionally substituted pyrenylmethylglycerol, or an optionally substituted heteroalicylic or optionally substituted heteroaromatic such as optionally substituted pyridyloxazole, optionally substituted pyrrole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R^{2*}, R³, and R^{3*} is a group P* which designates an internucleoside linkage to a preceding monomer, or a 2′/3′-terminal group. The substituents of R^{1*}, R^{4*}, R⁵, R^{6*}, R⁶, R^{6*}, R⁷, R^{7*}, R^{N}, and the ones of R², R^{2*}, R³, and R^{3*} not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C═Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxycarbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R^{1*}, R², R^{2*}, R³, R^{3*}, R^{4*}, R⁵, R^{5*}, R⁶ and R^{6*}, R⁷, and R^{7*} which are present and not involved in P, P* or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R^{1*}, R², R^{2*}, R³, R^{4*}, R⁵, R^{5*}, R⁶ and R^{6*}, R⁷, and R^{7*} which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆-alkyl)amino, carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g. acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

It is understood that references herein to a nucleic acid unit, nucleic acid residue, LNA monomer, or similar term are inclusive of both individual nucleoside units and nucleotide units and nucleoside units and nucleotide units within an oligonucleotide.

A “modified base” or other similar terms refer to a composition (e.g., a non-naturally occurring nucleobase or nucleosidic base), which can pair with a natural base (e.g., adenine, guanine, cytosine, uracil, and/or thymine) and/or can pair with a non-naturally occurring nucleobase or nucleosidic base. Desirably, the modified base provides a Tₘ differential of 15, 12, 10, 8, 6, 4, or 2° C. or less as described herein. Exemplary modified bases are described in EP 1 072 679 and WO 97/12896.

The term “chemical moiety” refers to a part of a molecule. “Modified by a chemical moiety” thus refer to a modification of the standard molecular structure by inclusion of an unusual chemical structure. The attachment of said structure can be covalent or non-covalent.

The term “inclusion of a chemical moiety” in an oligonucleotide probe thus refers to attachment of a molecular structure. Such as chemical moiety include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, PicoGreen, thiazole orange, Hoechst 33342, Ethidium Bromide, 1-O-(1-pyrenylmethyl)glycerol and Hoechst 33258. Other chemical moieties include the modified nucleobases, nucleosidic bases or LNA modified oligonucleotides.

“Oligonucleotide analog” refers to a nucleic acid binding molecule capable of recognizing a particular target nucleotide sequence. A particular oligonucleotide analogue is peptide nucleic acid (PNA) in which the sugar phosphate backbone of an oligonucleotide is replaced by a protein like backbone. In PNA, nucleobases are attached to the uncharged polyamide backbone yielding a chimeric pseudopeptide-nucleic acid structure, which is homomorphous to nucleic acid forms.

“High affinity nucleotide analogue” refers to a non-naturally occurring nucleotide analogue that increases the “binding affinity” of an oligonucleotide probe to its complementary recognition sequence when substituted with at least one such high-affinity nucleotide analogue. Commonly used analogues include 2′-O-methyl-modified nucleic acids (2′-OMe) (RNA, 2006, 12, 163-176), 2′-O-(2-methoxyethyl)-modified nucleic acids (2′-MOE) (Nucleic Acids Research, 1998, 26, 16, 3694-3699), 2′-Deoxy-2′-fluoro-β-D-arabinoic acid (FANA) (Nucleic Acids Research, 2006, 34, 2, 451-461), Cyclohexene nucleic acids (CeNA) (Nucleic Acids Research, 2001, 29, 24, 4941-4947), Hexitol nucleic acids (HNA) and analogs hereof (Nucleic Acids Research, 2001, 29, 20, 4187-4194), Intercalating Nucleic Acids (INA) (Helvetica Chimica Acta, 2003, 86, 2090-2097) and 2′-O,4′-C-Ethylene-bridged-Nucleic Acids (ENA) (Bioorganic and Medicinal Chemistry Letters, 2002, 12, 1, 73-76). Additionally, in the present context, the oligonucleotide mimic referred to as peptide nucleic acid (PNA) (Nielsen et al., Science 254; 1497-1500, 1991 and U.S. Pat. Nos. 5,539,082; 5,714,331; and 5,719,262) is considered a high affinity nucleotide analogue. A preferred high affinity nuceltodie analogue is LNA. A plurality of a combination of analogues may also be employed in an oligo of the invention.

As used herein, an oligo with an increased “binding affinity” for a recognition sequence compared to an oligo that includes the same sequence but does not include a nucleotide analog, refers to an oligo for which the association constant (Kₐ) of the recognition segment is higher than the association constant of the complementary strands of a double-stranded molecule. In a preferred embodiment, the association constant of the recognition segment is higher than the dissociation constant (K_{d}) of the complementary strand of the recognition sequence in the target sequence in a double stranded molecule.

Monomers are referred to as being “complementary” if they contain nucleo-bases that can form hydrogen bonds according to Watson-Crick base-pairing rules (e.g. G with C, A with T or A with U) or other hydrogen bonding motifs such as for example diaminopurine with T, 5-methyl C with G, 2-thiothymidine with A, inosine with C, pseudoisocytosine with G, etc.

The term “succeeding monomer” relates to the neighbouring monomer in the 5′-terminal direction and the “preceding monomer” relates to the neighbouring monomer in the 3′-terminal direction.

The term “target nucleic acid” or “target ribonucleic acid” refers to any relevant nucleic acid of a single specific sequence, e.g., a biological nucleic acid, e.g., derived from a patient, an animal (a human or non-human animal), a plant, a bacteria, a fungi, an archae, a cell, a tissue, an organism, etc. For example, where the target ribonucleic acid or nucleic acid is derived from a bacteria, archae, plant, non-human animal, cell, fungi, or non-human organism, the method optionally further comprises selecting the bacteria, archae, plant, non-human animal, cell, fungi, or non-human organism based upon detection of the target nucleic acid. In one embodiment, the target nucleic acid is derived from a patient, e.g., a human patient. In this embodiment, the invention optionally further includes selecting a treatment, diagnosing a disease, or diagnosing a genetic predisposition to a disease, based upon detection of the target nucleic acid.

“Target sequence” refers to a specific nucleic acid sequence within any target nucleic acid.

The term “stringent conditions”, as used herein, is the “stringency” which occurs within a range from about Tₘ−5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63: 378-383, 1969; and John, et al. Nature 223: 582-587, 1969.

### DETAILED DESCRIPTION OF THE INVENTION

siRNAs target mRNA sequences in a sequence specific manner by inducing mRNA degradation or in some cases inhibiting protein synthesis by blocking translation. However, off target gene silencing mediated by either strand of the siRNA can result in undesired changes in the expression of several genes and induce measurable phenotypes.

It is therefore desirable to have available reagents that can specifically block the effect of individual siRNAs or antisense molecules on single genes. One way to achieve this is by introducing into cells, such as by transfection, nucleic acid constructs that are homologous to the target sequence of the siRNA or antisense molecule and are able to block this target site thereby making it inaccessible to the regulatory siRNA or antisense molecule. If the specific blocking of the interaction of the siRNA to its target site reestablishes the phenotype of cells, which do not express the siRNA, no off-targeting effect is associated with the siRNA. However, it is of utmost importance that the blocking nucleic acid molecule can bind with high affinity compared to siRNA or antisense molecules, that it has high nuclease resistance, and very importantly that it does not induce antisense (e.g., RNaseH induction) effect on the target molecule.

High affinity nucleic acid analog (e.g., LNA) containing molecules have several advantages for this purpose:
Nucleic acid analogs can increase thermal stability allowing the blocking molecule to bind preferentially to the target site of the siRNA or antisense molecule, preferably to the siRNA or antisense molecule itself.
Nucleic acid analog containing molecules show increased stability compared to natural nucleic acid molecules either DNA or RNA
It has been shown that when employing nucleotide analogues such as LNAs for antisense molecules, a 5-8 nucleotide centrally located “gap” of un-modified DNA or RNA molecules is necessary to induce RNAseH mediated degradation of the target (Kurreck et al., Nucleic Acids Res. 30(9); 1911-1918, 2002).
Furthermore it has been demonstrated that LNA does not induce interferon response in in vivo administration.

Thus, nucleic acid molecules, which do not induce antisense (e.g., RNaseH induction) effects on the target molecule can be designed to block regulatory target sites for siRNA or antisense molecules.

Design Parameters for siRNA Blocking Oligos

One advantageous design principle would be to include, at least, interspaced nucleic acid analogues in the entire sequence, to prevent the formation of gapmers. Gaps should not exceed 4 nucleotides.

Another advantageous design feature would be to include in the blocking nucleic acid nucleic acid analogues in the 3′ and 5′ ends to enhance bio-stability and to decrease liability to intracellular nucleases.

In order to design siRNA target site blocking oligos, functional siRNAs need to be designed and their target sites identified and preferably validated.

In principle any region of the mRNA can be targeted by a siRNA. Several guidelines for helping researchers to design siRNAs that can effectively silence gene expression are available for those skilled in the art (see De Paula et al., RNA 13; 431-456, 2007 for a review) and a number of academic and commercially affiliated Web-based softwares have been developed to assist researchers in the identification of efficient siRNA sequences (Naito et al. Nucleic Acids Res. 32; W124-W129, 2004; Yuan et al., Nucleic Acids Res. 32; W130-W134, 2004). To ensure that the chosen siRNA sequence targets a single gene, a search of the selected target site sequence should be carried out against sequence databases such as the Smith-Waterman algorithm or the Basic Local Alignment Search Tool (BLAST) located at the National Center of Biotechnology Information Website. Potential targets can typically be validated by using luciferase reporters containing the target 3′UTR.

When the siRNA has been designed, a person skilled in the art will be able to also design a blocking oligo according to the present invention which binds to a region including a portion of the selected siRNA target site and a naturally occurring nucleic acid sequence adjacent to the selected siRNA target site.

Other design parameters for blocking oligos:

Blocking Oligos Block siRNA Target Binding Efficiently.

siRNA molecules can target mRNA sequences with both complete and incomplete homology between the siRNA sequence and target. For incomplete binding matches of positions 2-7 or 2-8 of the antisense strand, the so called seed sequence of the siRNA target site, is of key importance for the siRNA binding and effect (Bimingham) et al., Nature Methods 3(3); 199-204, 2006). The blocking oligo should preferably block at least 1-3 of the siRNA nucleotide binding events, more preferably 3-6 nucleotide binding events, also preferably selected from the seed sequence region.

To measure the effect of siRNA blocking oligonucleotides, a relative measure comparing the range between 1) the expression level of a given siRNA target nucleotide or resulting protein under an approximate maximum effect of a siRNA (e.g., given the effect of over-expression of the siRNA) and 2) the expression level of the siRNA target nucleotide or resulting protein without the siRNA present (e.g., in a cell not expressing the siRNA or by co-transfecting with a siRNA knockdown probe) with 3) the expression level of the siRNA target nucleotide or resulting protein under an approximate maximum effect of a siRNA (e.g., over-expression of the siRNA) and in the presence of a given concentration of a siRNA blocking oligo targeting the same siRNA target nucleotide. For example, if the expression level of the siRNA target nucleotide or resulting protein is changed by 50% of the range between 1) and 2) by addition of the siRNA target site blocking oligo of a given concentration under approximate maximum effect of the siRNA, the target site blocking oligo will have blocked 50% of the activity of the siRNA at that given concentration.

The amount of the target may be reflected in the relative expression level of the siRNA target nucleotide (e.g., a messenger RNA as determined by QPCR or northern blot or similar technologies) or in the relative expression level of the translational product (protein, as determined by e.g. Western blotting or by measuring the enzymatic or catalytic activity of the resulting protein (e.g. as lucifierase activity in case of the luciferase enzyme)) of the siRNA target nucleotide.

A miRNA with a corresponding verified binding site can be selected for feasibility experiments. Reporter vector (A) is made with the binding site of the miRNA cloned into the 3′UTR of luciferase gene. Another reporter vector (B) is made with a sequence which is exactly complementary to the miRNA and cloned into the 3′UTR of luciferase gene. This construct transfected into a cell line will model miRNA acting as a siRNA because of exact complimentarity.

The reporter vector (A) transfected into a cell line which do not express the selected miRNA will be expected to show expression of lucifirase. The co-transfection of reporter vector (A) and the miRNA is expected to show a reduction in the expression of lucifirase compared to cells transfected by vector alone.

The reporter vector (B) when transfected into a cell line which do not express the selected miRNA will show expression of lucifirase. The co-transfection of reporter vector (B) and the miRNA will be expected to show reduction of expression of lucifirase based on siRNA type activity compared to cells transfected by vector alone.

The reporter vector (B) is transfected into a cell line expressing the miRNA (acting as a siRNA) and a blocking oligo of the present invention and an increase in expression of lucifirase to the initial level should be expected.

The Blocking Oligo Desirably Targets a Single siRNA Binding Site, Specific to a Particular mRNA.

It is preferable that the blocking oligo can be designed to target only a single specific mRNA. Since each siRNA may target multiple mRNAs by off-targeting, the siRNA target site and off-target sites in different mRNAs may be very similar, hence allowing a blocking oligo designed to target one specific site, to also block other off-target sites. This can be avoided by designing the blocking oligo to cover part of the adjacent non-target site mRNA sequence, since this sequence will likely be specific for the mRNA in question. In a preferred embodiment, the blocking oligo recognizes a portion of the 3′ end of the target site and a longer sequence 3′ adjacent to the target site. Furthermore, the oligo designed can be compared to a database comprising the complete transcriptome (e.g., by a BLAST search). The oligo sequence preferably should not occur more than once in such a database. Preferably, similarity with other sites differing by fewer than 2 nucleotides in identity is avoided.

The Blocking Oligo Desirably Does Not Target the Double Stranded RNA or Small Hairpin RNA Molecule which is to be Processed into siRNA.

The sequence of the blocking oligo will be at least partially identical to the targeting siRNA sequences. Since siRNAs may be produced from longer processed double stranded RNA molecules or small hairpin RNA structures involving the sequence of the siRNA, care should be taken to avoid that a siRNA target site blocking oligo comprising the complete siRNA target sequence blocks the precursor RNA molecule and hence eliminates the production of the specific siRNA.

Several siRNA Binding Events May Induce Degradation of a Specific mRNA and Hence Several siRNA Blocking Oligos may be Required to Protect a Specific mRNA from Degradation.

Accessible siRNA target sites may be rare in some mammalian mRNAs. However, more effective gene silencing can be achieved by targeting different segments of the same transcript simultaneously with two or more siRNAs against different sites of the same mRNA (Ji et al., FEBS Lett. 552; 247-252, 2003). Thus, in certain embodiments the present invention provides for the administration of more than one oligonucleotide as described herein for blocking of more than one siRNA target sites of a particular target mRNA.

### Resistance to Degradation

It has been shown (Kurreck et al., Nucleic Acids Research 30(9); 1911-1918, 2002) that LNA/DNA mixmers do not induce significant RNase H cleavage. A gap in a chimeric LNA/DNA oligonucletoide is needed to recruit RNase H and a DNA stretch of 7-8 nucleotides was found to provide full activation of RNase H. For gapmers with 2′-O-methyl modifications a shorter stretch of only six deoxy monomers is sufficient to induce efficient RNase H cleavage.

In a preferred embodiment, the single stranded oligonucleotide according to the invention does not mediate RNase H based cleavage of a complementary single stranded RNA molecule.

EP 1 222 309 provides in vitro methods for determining RNase H activity, which may be used to determine the ability to recruit RNase H. A compound is deemed essentially incapable of recruiting RNAse H if, when provided with the complementary RNA target, and RNase H, the RNase H initial rate, as measured in pmol/l/min, is less than 20%, such as less than 10%, such as less than 5%, or less than 1% of the initial rate determined using the equivalent DNA only oligonucleotide using the methodology provided by Example 91-95 of EP 1 222 309.

A compound is deemed capable of recruiting RNase H if, when provided with the complementary RNA target it has an initial rate, as measured in pmol/l/min, of at least 1% such as at least 5%, such as at least 10% or less than 20% of the equivalent DNA only oligonucleotide using the methodology provided by Example 91-95 of EP 1 222 309.

### Methods for Performing RNA Interference.

Any method known in the art can be used for carrying out RNA interference. In one embodiment, gene silencing is induced by presenting the cell with the siRNA, mimicking the product of Dicer cleavage (see, e.g., Elbashir et al., 2001, Nature 411, 494-498; Elbashir et al., 2001, Genes Dev. 15, 188-200, all of which are incorporated by reference herein in their entirety). Synthetic siRNA duplexes maintain the ability to associate with RISC and direct silencing of mRNA transcripts, thus providing researchers with a powerful tool for gene silencing in mammalian cells. siRNAs can be chemically synthesized, or derived from cleavage of double-stranded RNA by recombinant Dicer.

Another method is to introduce a double stranded DNA (dsRNA) for gene silencing is shRNA, for short hairpin RNA (see, e.g., Paddison et al., 2002, Genes Dev. 16, 948-958; Brummelkamp et al., 2002, Science 296, 550-553; Sui, G. et al. 2002, Proc. Natl. Acad. Sci. USA 99, 5515-5520, all of which are incorporated by reference herein in their entirety). In this method, a desired siRNA sequence is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells both in vitro and in vivo, e.g., in animals (see, McCaffrey et al. 2002, Nature 418, 38-39; Xia et al., 2002, Nat. Biotech. 20, 1006-1010; Lewis et al., 2002, Nat. Genetics 32, 107-108; Rubinson et al., 2003, Nat. Genetics 33, 401-406; Tiscornia et al., 2003, Proc. Natl. Acad. Sci. USA 100, 1844-1848, all of which are incorporated by reference herein in their entirety).

In yet another method, siRNAs can be delivered to an organ or tissue in an animal, such a human, in vivo (see, e.g., Song et al. 2003, Nat. Medicine 9, 347-351; Sorensen et al., 2003, J. Mol. Biol. 327, 761-766; Lewis et al., 2002, Nat. Genetics 32, 107-108, all of which are incorporated by reference herein in their entirety). In this method, a solution of siRNA is injected intravenously into the animal. The siRNA can then reach an organ or tissue of interest and effectively reduce the expression of the target gene in the organ or tissue of the animal.

### Methods for Determining Biological State and Biological Response.

This invention provides methods comprising determining response profiles, such as changes of phenotypes, of specific blocking of siRNA perturbation. The measured responses can be measurements of cellular constituents in a cell or organism or responses of a cell or organism to a specific blocking of siRNA perturbation. The cell sample can be of any organism in which RNA interference can occur, e.g., eukaryote, mammal, primate, human, non-human animal such as a dog, cat, horse, cow, mouse, rat, Drosophila, C. elegans, etc., plant such as rice, wheat, bean, tobacco, etc., and fungi. The cell sample can be from a diseased or healthy organism, or an organism predisposed to disease. The cell sample can be of a particular tissue type or development stage and subjected to a particular siRNA perturbation. One of skill in the art would appreciate that this invention is not limited to the following specific methods for measuring the phenotypes, such as expression profiles and responses, of a biological system.

### Transcript Assays Using Microarrays.

One aspect of the invention provides polynucleotide probe arrays for simultaneous determination of the expression levels of a plurality of genes and methods for designing and making such polynucleotide probe arrays. The expression level of a nucleotide sequence in a gene can be measured by any high throughput techniques. However measured, the result is either the absolute or relative amounts of transcripts or response data, including but not limited to values representing abundance ratios. Preferably, measurement of the expression profile is made by hybridization to transcript arrays, such as described in PCT patent application no. WO 2005/18534.

The relative abundance of an mRNA and/or an exon expressed in an mRNA in two cells or cell lines is scored as different (i.e., the abundance is different in the two sources of mRNA tested) or as identical (i.e., the relative abundance is the same). As used herein, a difference between the two sources of RNA of at least a factor of about 25% (i.e., RNA is 25% more abundant in one source than in the other source), more usually about 50%, even more often by a factor of about 2 (i.e., twice as abundant), 3 (three times as abundant), or 5 (five times as abundant) is scored as different. Present detection methods allow reliable detection of difference of an order of about 3-fold to about 5-fold, but more sensitive methods are expected to be developed.

Other Methods of Transcriptional State Measurement.

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent O 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) that are generated at known positions relative to a defined mRNA end (see, e.g., Velculescu, 1995, Science 270:484-487).

### Measurement of Other Aspects of the Biological State.

In various embodiments of the present invention, aspects of the biological state other than the transcriptional state, such as the translational state, the activity state, or mixed aspects can be measured to produce the measured signals to be analyzed according to the invention. Thus, in such embodiments, gene expression data may include translational state measurements or even protein expression measurements. In fact, in some embodiments, rather than using gene expression interaction maps based on gene expression, protein expression interaction maps based on protein expression maps are used.

### Embodiments Based on Translational State Measurements.

Measurement of the translational state may be performed according to several methods. For example, whole genome monitoring of protein (i.e., the “proteome,” Goffeau et al., 1996, Science 274:546-567; Aebersold et al., 1999, Nature Biotechnology 10:994-999) can be carried out by constructing a microarray in which binding sites comprise immobilized, preferably monoclonal, antibodies specific to a plurality of protein species encoded by the cell genome (see, e.g., Zhu et al., 2001, Science 293:2101-2105; MacBeath et al., 2000, Science 289:1760-63; de Wildt et al., 2000, Nature Biotechnology 18:989-994). Preferably, antibodies are present for a substantial fraction of the encoded proteins, or at least for those proteins relevant to the action of an siRNA of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In a preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated and measured by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of proteins: A Practical Approach, IRL Press, New York; Shevchenko et al., 1996, Proc. Natl. Acad. Sci. USA 93:1440-1445; Sagliocco et al., 1996, Yeast 12:1519-1533; Lander, 1996, Science 274:536-539; and Beaumont et al., Life Science News 7, 2001, Amersham Pharmacia Biotech. The resulting electropherograms can be analyzed by numerous techniques, including mass spectrometric techniques, Western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to an siRNA and/or a blocking oligo of the invention, or in cells modified by, e.g., deletion or over-expression of a specific gene.

### Embodiments Based on Other Aspects of the Biological State.

The methods of the invention are applicable to any cellular constituent that can be monitored. In particular, where activities of proteins can be measured, embodiments of this invention can use such measurements. Activity measurements can be performed by any functional, biochemical, or physical means appropriate to the particular activity being characterized. Where the activity involves a chemical transformation, the cellular protein can be contacted with the natural substrate(s), and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, phenotype data may be formed of mixed aspects of the biological state of a cell. Phenotype data can be constructed from, e.g., changes in certain mRNA abundances, changes in certain protein abundances, and changes in certain protein activities.

Determining and Modulating the Functional Role of siRNAs

Since siRNAs has been found to also elicit their effect through incomplete binding to target nucleotide sequences (the siRNA acts as a miRNA), bioinformatically predicting the target nucleotides (e.g., mRNAs) of a given siRNA based on its sequence alone is not trivial and may not provide evidence that interaction is occurring in vivo. One way of experimentally investigating the interaction between a siRNA and its target is to inactivate the siRNA in question (e.g., by providing a complementary knock-down oligo). However, each siRNA may target off-target transcripts in the cell. Hence, inactivating a specific siRNA in a cell may not directly provide evidence for interaction between a specific siRNA and a target nucleic acid (e.g., mRNA), since potential effects may be elicited by interactions between the siRNA and undesired targets in the cell.

A challenge in siRNA research is therefore to establish evidence that an interaction occurs between a siRNA and a prediction siRNA target site in a target nucleic acid (e.g., mRNA). By providing a method by which to specifically block a particular siRNA target site, the present invention provides a solution to study the specific interaction between a siRNA and its target site.

The introduction of a siRNA blocking oligo of the present invention which exactly match (or most of) the entire proposed siRNA binding site in a cell expressing would protect the mRNA from being degraded by siRNA activity even when the siRNA is expressed in the cell at a functional concentration. Such a blocking oligo may be able to bind to off-target sites on several mRNAs but could in a preferred embodiment be used for detecting immunoresponse and other non-siRNA pathway associated effects induced by the siRNA.

Another aspect of the present invention is a blocking oligo which will partially bind to an expected siRNA binding site as well as an adjacent sequence of the desired mRNA target for that siRNA. Preferably, this blocking oligo also prevents binding of the siRNA to the desired mRNA target and will not bind to potential off-target sites on other mRNAs. Thus, this gene-specific siRNA blocking oligo may be used for investigating off-target effect of the siRNA as described herein.

Modulating siRNA Interactions for Specific Target Nucleotides to Detect Off-Targeting Effect.

Vigorous in vitro and in vivo proof-of concept studying has showed that practically every human disease with a gain-of-function genetic lession can become a target for therapeutic RNAi. For its therapeutic applications, siRNA must not cause any unintended effects, such as off-target effects, immune response activation and/or non-specific gene silencing, other than sequence-specific gene silencing. However, as described, each siRNA may have up to multiple undesired off-target nucleic acids (e.g., mRNAs) in the cell. In strategies to design therapeutic siRNAs, a challenge in siRNA research has therefore been to establish methods for determining whether treatment with a siRNA is associated with off-target effects. By providing a method by which to specifically block a particular siRNA target site in a particular target nucleic acid, the present invention provides a solution for developing specific therapeutic siRNAs.

It has been proposed that siRNA off-target effects can be explained by miRNA-like activity of siRNA in RISC complex. In this situation the 5′ end of the siRNA can serve as seed sequence. Therefore, in a preferred embodiment the siRNA blocking oligo binds to an mRNA sequence complimentary to the 3′ end of the siRNA.

A method of identifying siRNA off-target effects using the siRNA target site blocking oligos could be to apply a similar analysis approach as described in (Jackson et al. Nat. Biotechnol. 21; 635-637; 2003, PCT patent application no. WO 2005/18534). If a siRNA is designed to target a specific gene, an experiment to test for off-target effects could be to co-transfect a cell line with a target site blocking oligo together with the specific siRNA. If the target site blocking oligo is efficient in reducing the interaction between the siRNA and the intended target, to reduce the degradation of said target significantly (e.g. reduce more than 80% of the effect), then one would expect to see no or very few gene-regulation effects of the siRNA oligo, compared to a cell line transfected with only a scrambled control oligo, unless the siRNA target has direct off-target effects on other oligos. Hence, analysis of microarray-based gene expression data could reveal potential off-target effects on other genes than the intended target. Alternatively, one could decide to design multiple siRNA oligos to the specific target, and select the siRNA having the least gene regulations when transfected with the corresponding target site blocking oligo, compared to a cell line transfected with only a scrambled control oligo. In addition, control experiments should be conducted to test for any effect of the blocking oligo alone by transfecting a cell line with the blocking oligo alone.

To evaluate the presence of an identified off target effect, the primary target gene as well as a potential off-target gene could be cloned in separate vectors under the control of the same type of promoter. Hence, expression from one construct should not affect the transcription of other construct. The two or more constructs should be co-transfected into a relevant cell line, and subsequently transfected with the siRNA targeting the primary gene target. One can then use gene expression analysis (eg. real time PCR such as TaqMan or microarrays such as Affymetrix® arrays) to evaluate effect on transcript levels from the two constructs. Also, protein levels can be measured by Western blotting to evaluate effects on protein expression. If transfection of a siRNA targeting one construct but not intentionally targeting the other gene construct only affects the target, no off target effects are observed. If the other construct is significantly reduced by the transfection of a siRNA targeting one construct but not intentionally targeting the other gene construct, it is likely that the siRNA in question has off-target effects on the other gene construct. As a control experiment, one can design a second siRNA targeting the primary target but having no or very little sequence similarity with the potential off target gene, and test whether the siRNA ceases to produce off-target effects, which would indicate that the off-target effects were caused by the siRNA.

### EXAMPLES

The invention will now be further illustrated with reference to the following examples. It will be appreciated that what follows is by way of example only and that modifications to detail may be made while still falling within the scope of the invention.

### Example 1

### Synthesis, Deprotection and Purification of LNA-Substituted Oligonucleotides

LNA-substituted oligos were prepared on an automated DNA synthesizer (Expedite 8909 DNA synthesizer, PerSeptive Biosystems, 0.2 μmol scale) using the phosphoramidite approach (Beaucage and Caruthers, Tetrahedron Lett. 22: 1859-1862, 1981) with 2-cyanoethyl protected LNA and DNA phosphoramidites, (Sinha, et al., Tetrahedron Lett. 24: 5843-5846, 1983). CPG solid supports derivatised with a suitable quencher and 5′-fluorescein phosphoramidite (GLEN Research, Sterling, Va., USA). The synthesis cycle was modified for LNA phosphoramidites (250s coupling time) compared to DNA phosphoramidites. 1 H-tetrazole or 4,5-dicyanoimidazole (Proligo, Hamburg, Germany) was used as activator in the coupling step.

The probes were deprotected using 32% aqueous ammonia (1 h at room temperature, then 2 hours at 60° C.) and purified by HPLC (Shimadzu-SpectraChrom series; Xterra™ RP18 column, 10 μm 7.8×150 mm (Waters). Buffers: A: 0.05M Triethylammonium acetate pH 7.4. B. 50% acetonitrile in water. Eluent: 0-25 min: 10-80% B; 25-30 min: 80% B). The composition and purity of the probes were verified by MALDI-MS (PerSeptive Biosystem, Voyager DE-PRO) analysis.

### Example 2

### Design of Blocking Molecules

Previous experiments using antagonizing oligos have demonstrated that important parameters for successful design are probe Tm and oligo self-annealling. If oligonucleotide Tm is too low, the efficiency is generally poor, maybe due to the oligo being removed from the target sequence by endogenous helicases. If Tm is too high, there is an increased risk that the oligo will anneal to partly complementary sites possibly leading to unspecific effects. With respect to selfannealing (autocomplementarity) of the probe, a low selfannealing score (reflecting stability of the autoduplex) is favorable. Previous results have shown that probes exceeding a selfannealing score of about 45 often show very low potency or are completely nonfunctional. The effect of a high selfannealing score is a stable autoduplex which obviously sequestrates large amounts of probes, preventing the probe from interacting with its target sequence. To avoid high stability of the autoduplex, it is important to prevent LNA nucleotides in stretches of autocomplementary sequences. This may be acheived by an iterative approach in which the starting point is an oligonucleotide sequence consisting of only LNA monomers. This oligonculeotide is then put through a selfannealing scoring program (Exiqon website) that also identifies nucleotides participating in duplex formation. Next, one or more of these nucleotides are substituted with DNA, and the process is repeated, again substituting LNAs participating in duplex formation with DNA thereby gradually reducing selfannealing score. When reaching an appropriate Tm and selfannealing score, the process is stopped. The process is repeated for sequences spanning various regions of the target sequence to find optimal selfannealing scores and Tms.

An additional requirement in an oligonucleotide design process was the preference of LNA nucleotides in the terminals of the oligonucleotide. This was done to preserve biostability of the oligo, thereby improving duration of the biological response.

### Example 3

### Blocking of miR-21 Target Binding Reporter Assay

TABLE 1

Oligonucleotides and sequences used in the reporter assay:
Oligonucleotide nameOligonucleotide sequence

| | | |
|---|---|---|
| Anti-21target Tm 73 | 5′-TAGmCTTATmCagAmCTGa-3′ | (SEQ ID NO: 16) |
| Anti-21target Tm 70 | 5′-TAGmCTTATmCagAmCtGa-3′ | (SEQ ID NO: 17) |
| Anti-2ltarget Tm 75 | 5′-TAGmCTTatmCAgAmCtgATg-3′ | (SEQ ID NO: 18) |
| Antitarget control Tm75 | 5′-AAmCTagTgmCgmCAgmCTTt-3′ | (SEQ ID NO: 19) |
| Antitarget control Tm74 | 5′-AAmCTAgTgmCgmCAgmCt-3′ | (SEQ ID NO: 20) |
| Antitarget control Tm71 | 5′-AAmCTagTgmCgmCAgmCt-3′ | (SEQ ID NO: 21) |
| 2′OMe anti-21 target | 5′-tagcttatcagactgatg-3′ | (SEQ ID NO: 22) |
| 2′OMe control | 5′-aactagtgcgcagcttt-3′ | (SEQ ID NO: 23) |

a) Oligo nucleotide name and sequence. The LNA monomers are uppercase letters, mC is LNA methyl cytosine, DNA monomers are lowercase letters, and 2′OMe monomers are bold lower letters. For the LNA containing oligonucleotides the name indicates the predicted Tm according to LNA-DNA Tm prediction tool (Tolstrup et al., Nucleic Acids Res 31(13; 3758-3762, 2003).

LNA containing oligonucleotides were obtained from the TIB MOLBIOL, whereas 2′OMe was obtained from the DNAtechnology. All oligonucleotides were HPLC purified, and correct molecular mass was verified using mass spectroscopy.

Design of Oligonucleotides

LNA oligonucleotides were designed as described in Example 2. To investigate the effect of the predicted Tm and the inhibitory efficiency of the LNA containing oligonucleotides, three different oligonucleotides with various Tms were designed to be perfectly complementary to the miR-21 target site in the miR-21 reporter vector (pMIR-21) resulting in predicted Tms of 73° C., 70° C., 75° C. Likewise three control oligonucleotides were designed and synthesized with similar predicted Tm (see table 1) but complementary to a region of the 3′UTR immediately adjacent to the miR-21 target site. This region is also present in the pMIR-16 control vector. The 2′OMe antitarget blocking and control oligonucleotides were designed to contain the same sequence as the LNA containing Anti-21target Tm 75 and Antitarget control Tm75 oligonucleotides as shown in table 1.

b) miRNA Reporter Constructs

The pMIR-21 was constructed by inserting a miR-21 complementary sequence in the 3′UTR of the pMIR-REPORT (Ambion) containing the firefly luciferase reporter gene. This was done by annealing oligonucleotide I (A: 5′-AAT GCA CTA GTT CAA CAT CAG TCT GAT AAG CTA GCT CAG CAA GCT TAA TGC- 3′; SEQ ID NO:24) and II (B: 5′-GCA TTA AGC TTG CTG AGC TAG CTT ATC AGA CTG ATG TTG MC TAG TGC ATT-3′; SEQ ID NO:25). This fragment and the pMIR-REPORT vector were then digested with SpeI and HindIII, and the fragment was subsequently cloned into the SpeI and HindIII sites of pMIR-REPORT vector using standard techniques, thereby generating pMIR-21. The pMIR-16 was constructed using the same procedure but with the following DNA oligonucleotides for the insert: I (A: 5′-AAT GCA CTA GTC GCC AAT ATT TAC GTG CTG CTA GCT CAG CAA GCT TAA TGC-3′; SEQ ID NO:26) and II (B: 5′-GCA TTA AGC TTG CTG AGC TAG CAG CAC GTA AATA TGG CGA CTA GTG CAT T-3′; SEQ ID NO:27).

c) Reporter Assays

HeLa and MCF7 cells were propagated in Dulbecco's Modified Eagle's Minimal Essential Medium (DMEM) with Glutamax™ (Invitrogen) and supplemented with 10% foetal bovine serum (FBS). On the day prior to transfection cells were seeded in 96-well plates (Corning) at a density of 7000 cells/well. Cells were transfected using Xtreme Gene siRNA (Roche), with 70 ng/well of pMIR-21 reporter and 30 ng/well of the pGL4.73 Renilla (Promega) reporter plasmid for normalisation. Where indicated transfection mix also contained oligonucleotides resulting in a final concentration of 10 nM, 20 nM and 50 nM.

After 3-4 h, media with transfection mix was removed and cells were washed four times in PBS and supplemented with fresh media. Luciferase activities (Firefly and Renilla) were measured 24 h later using the Dual Glow Luciferase kit (Promega) on a BMG Optima luminometer.

For the MCF7 cells, experiments were carried out as above, however these cells were propagated in Roswell Park Memorial Institute medium (RPMI) 1640 with Glutamax™ (Invitrogen) and supplemented with 10% FBS. Cells were seeded to 15000 cell/well on the day prior to transfection and left for 48 h before measuring luciferase activity.

After luminescence measurements relative light units (RLU) were corrected for background and firefly luminescence (FL) was normalised to Renilla luminescence (RL). Data presented in the diagram show normalised FL activity as a function of oligonucleotide concentration and cell line.

Results

To measure the effect of siRNA blocking oligonucleotides, a luciferase based miR-21 sensor reporter was constructed. This reporter harbours a sequence fully complementary to hsa-miR-21. When the reporter mRNA is recognized by a miR-21 containing RISC complex, the luciferase encoding mRNA is cleaved and subsequently degraded. This may be similar to the siRNA mediated silencing process since the there is perfect complementarity between target and the mir-21 sequence. The luciferase expression level thereby reflects the endogenous level and activity of miR-21. Likewise an identical control vector harbouring a 22 nt miR-16 complementary sequence was also constructed (pMIR-16).

In this line of experiments the pMIR-21 plasmid and control plasmid pMIR-16 were cotransfected with the siRNA-Target site blocking oligonucleotides (table 1), and reporter activity was subsequently measured.

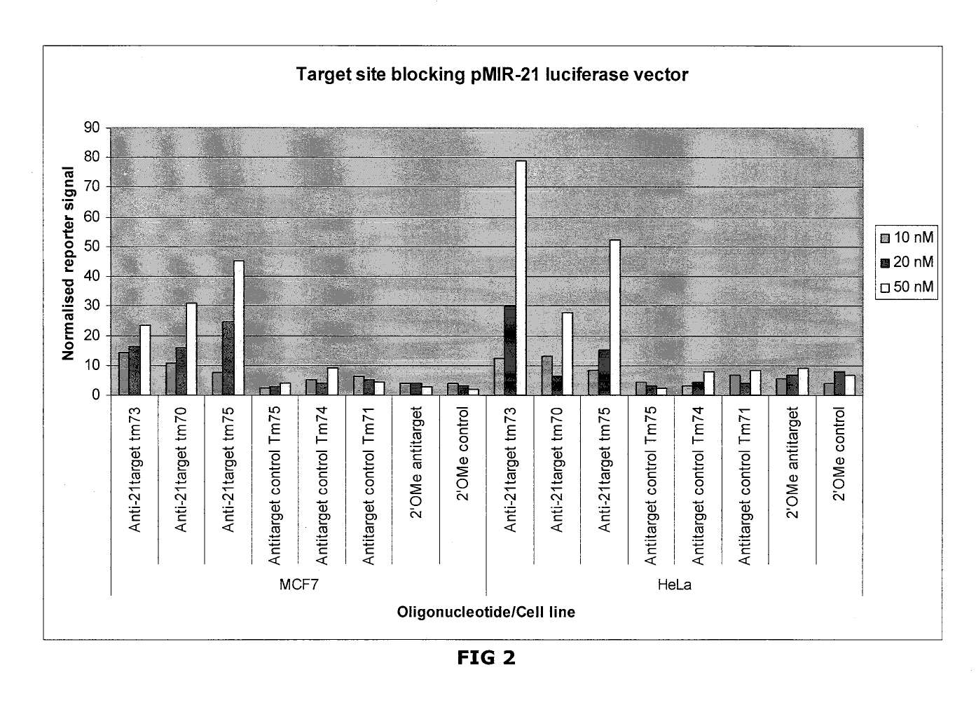

Reporter data show that when co-transfected with miR-21 reporter plasmid all LNA containing oligonucleotides complementary to the miR-21 target site resulted in increased reporter activity (FIG. 2). Relative to the control oligonucleotides, reporter activity increased as much a 10-fold with a strong dose response not reaching saturation at 50 nM oligonucleotide concentration. None of the control oligonucleotides showed any significant effect on reporter activity despite being complementary to an adjacent 3′UTR sequence. This effect was evident for all three pairs of target site blocking and control oligonucleotides and was apparent in both MCF7 and HeLa cells.

In a control experiment, the miR-21 target site blocking oligonucletides and controls were cotransfected with the pMIR-16 reporter carrying a miR-16 complementary sequence whose activity is affected by the miR-16 expression level in the cell lines. Thus, this reporter is not a target for the miR-21, and miR-21 target site blocking oligonucleotides; however the vector is complementary to the control oligonucleotides targeting a 3′UTR sequence adjacent to the miR-target site.

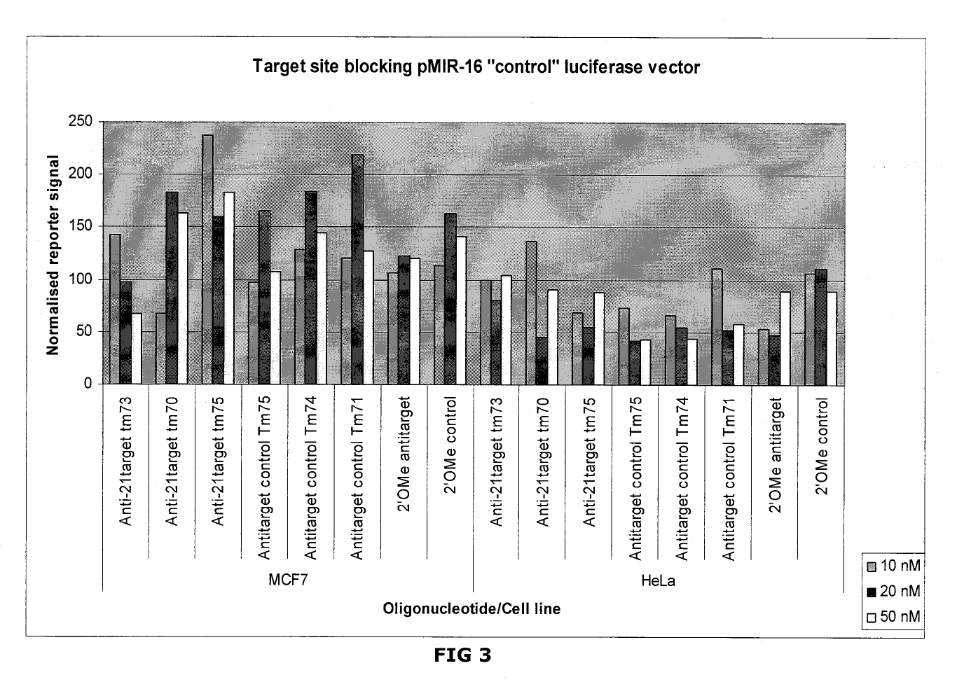

In these experiments (FIG. 3), reporter activity is only slightly affected by cotransfection with the miR-21 complementary oligonucleotides and shows no dose response curve in either cell line, demonstrating that no nonspecific sequence effect is generated by the miR-21 target site blocking oligonucleotides.

To address the effect of LNA oligonucleotides relative to 2′OMe modified oligonucleotides, a similar experiment was carried out using 2′OMe modified oligonucleotides as miR-21 target site blockers. Both miR-21 targeting and control oligonucleotides were designed to target identical sequence as the LNA containing oligonucleotides (see table 1).

The reporter results (FIG. 2) show that a significant effect of reporter activity was not observed in either HeLa or MCF7 cell lines, indicating that 2′OMe oligonucleotides were not ideally suitable for conditions required for blocking access of RISC-miRNA complex to the target transcripts. This may have to do with lower duplex stability of the 2′OMe and RNA compared to LNA RNA duplexes or to the lower biostability of 2′OMe modified oligonucleotides relative to LNA containing oligonucleotides (Grünweller et al., Nucleic Acids Res. 31(12); 3185-3193, 2003).

All together these experiments demonstrate sequence specific regulation of the reporter activity of the LNA containing oligonucleotides both on the level of oligonucleotide sequence and vector sequence. The effect is strong in both HeLa and MCF7 cell lines, both known to express high levels of miR-21 and miR-16 (Blower et al., Mol Cancer Ther 6(5); 1483-1491, 2007 and Meister et al., RNA 10(3); 544-550, 2004). This demonstrates the suitability of LNA modified oligonucleotides for a siRNA target site blocking approach.

### Example 4

### Blocking of Murine miR-181a Target Binding Reporter Assay

To measure the effect of siRNA blocking oligonucleotides, a luciferase based miR-181a sensor reporter was constructed. This reporter harbours a sequence fully complementary to hsa-miR-181a. When the reporter mRNA is recognized by a miR-181a containing RISC complex, the luciferase encoding mRNA is cleaved and subsequently degraded. This may be similar to the siRNA mediated silencing process since the there is perfect complementarity between target and the mir-181a sequence. The luciferase expression level thereby reflects the cellular level and activity of miR-181a.

Design of LNA Oligonucleotides

LNA oligonucleotides were designed as described. To investigate the effect of the predicted Tm and the inhibitory efficiency of the LNA containing oligonucleotides, seven different oligonucleotides with various sequence overlap and Tms were designed to be perfectly complementary to the miR-181a target site and surrounding sequence in the miR-181a reporter vector resulting in predicted Tms as indicated in table 2.

Reporter Assays:

HeLa S3 Cells Grown in DMEM (Invitrogen)/10% FCS (PAA)/penicillin & streptomycin (Invitrogen)/plasmocin (Invivogen) were transfected with Firefly luciferase reporter vector with a tandem repeat recognition site for miR181 present in the 3′ UTR, together with a puromycin resistance plasmid.

HeLa S3 cells do not express endogenous mir-181. For this reason it was possible to isolate stable cell populations expressing the reporter constructs Transfected cells were cultured were selected during three weeks with 1 μg/ml puromycin, and assayed for stable luciferase production.

For luciferase assays, stable cell lines expressing the miR-181 luciferase reporter were plated at 20 000 cells/well on 48 well culture plates, and immediately transfected with a miR-181a mimic (sense: aacauucaacgcugucggugagu (SEQ ID NO:8), antisense: caccgaccguugacuguacc (SEQ ID NO:9)), or a control irrelevant mimmick (sense: acuuaaccggcauaccggcdTdT (SEQ ID NO:10), gccgguaugccgguuaagudTdT (SEQ ID NO:11)) at 50 nM, using Lipofectamine 2000 (Invitrogen). 24 hours later, the cells were transfected with LNA-modified antisense oligonucleotides or TSB inhibitory molecules at 1, 5, 25, and 50 nM final. Luciferase activity was measured 48 h later (or 72 h after cell plating), and was normalized by total cell number using the quantitative WST1 kit (Roche). Transfection with the mir-181 mimic but not the control mimic results in strong repression of luciferase activity.

Results:

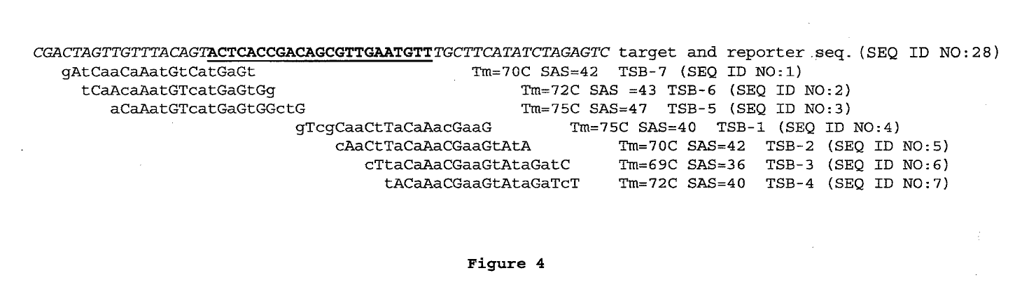

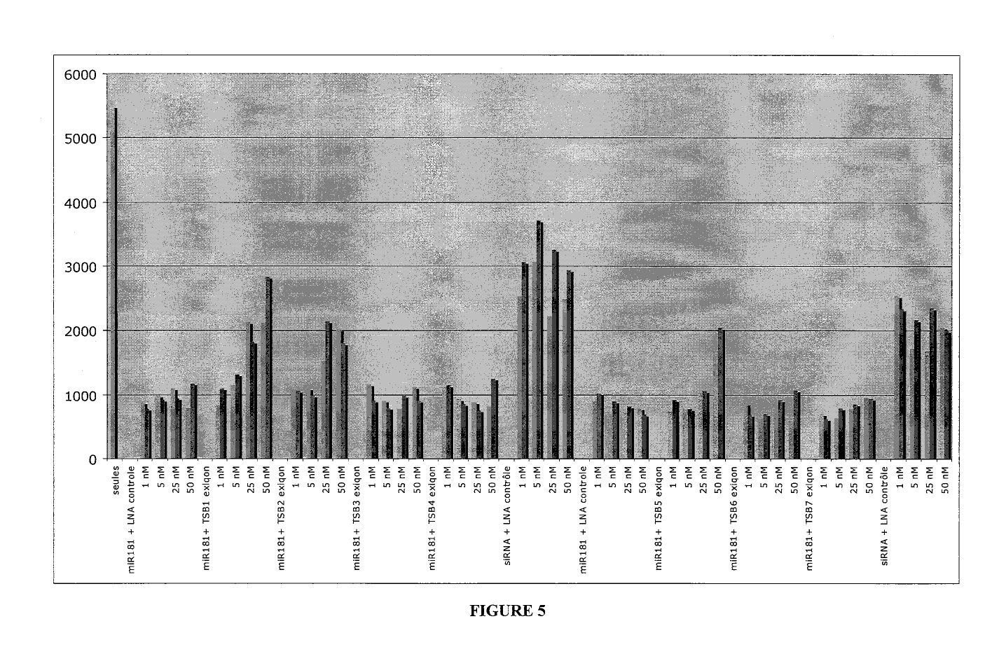

Reporter data show that LNA containing oligonucleotides TSB-1, TSB-2 and TSB-5 partially complementary to the miR-181a target site resulted in almost completely derepressed reporter activity (FIGS. 4 and 5). Relative to the control oligonucleotides, reporter activity increased as much a 3-fold with a strong dose response reaching saturation at 50 nM oligonucleotide concentration. None of the control oligonucleotides showed any significant effect on reporter activity. This effect was evident for all three (TSB-1, TSB-2 and TSB-5) target site blocking oligonucleotides in HeLa cells.

As seen from FIG. 4, the oligos with best effect tend to overlap the target site more. This suggests that the TSB oligos derepress luciferase expression by hybridizing to the 3′UTR and thereby preventing the mir-181 RISC complex from binding to the mir-181 target site by sterical hindrance.

All references, patents, and patent applications cited herein are hereby incorporated by reference.

Other embodiments are in the claims.

1. A nucleic acid binding to a region comprising a portion of a naturally occurring target site of a siRNA and, optionally, to a naturally occurring nucleic acid sequence adjacent to said target site of a siRNA.

2. The nucleic acid of claim 1 comprising at least one high affinity nucleic acid analog.

3. The nucleic acid of claim 2, wherein said at least one high affinity nucleic acid analog is LNA.

4. The nucleic acid of claim 1, wherein said nucleic acid binds to the 3′ end of said target site.

5. The nucleic acid of claim 1, wherein said nucleic acid binds to the 5′ end of said target site.

6. The nucleic acid of claim 1, wherein said nucleic acid has a length from 5-30 nucleotides.

7. The nucleic acid of claim 1, wherein binding of said nucleic acid to said region reduces the binding of said siRNA to said region.

8. The nucleic acid of claim 7, wherein said binding of said nucleic acid to said region reduces the binding of said siRNA to said region by at least 50%.

9. The nucleic acid of claim 1, wherein said nucleic acid is RNase resistant.

10. The nucleic acid of claim 1, wherein said nucleic acid comprises up to 80% of said at least one high affinity nucleic acid analog or said at least one high affinity nucleic acid analog in combination with one or more additional analogs.

11. The nucleic acid of claim 1, wherein said nucleic acid binds to 100% of said target site.

12. The nucleic acid of claim 1, wherein said nucleic acid binds to said region with a lower Kd than said siRNA.

13. The nucleic acid of claim 1, wherein at least 10% of said nucleic acid is not complementary to said siRNA.

14. The nucleic acid of claim 1, wherein said nucleic acid has an increase in binding affinity to said region as determined by an increase in Tm of at least 2° C., compared to the naturally occurring RNA complement of said region.

15. The nucleic acid of claim 1, wherein said siRNA binds to more than one target in a genome, and wherein said naturally occurring nucleic acid sequence adjacent to said target site differs by three or more nucleotides from other such sequences.

16. The nucleic acid of claim 1, wherein said nucleic acid does not prevent production of said siRNA from its precursor dsRNA or shRNA.

17. The nucleic acid of claim 1, wherein said nucleic acid is complementary to at least two nucleotides of said target site.

18. The nucleic acid of claim 1, wherein said nucleic acid is complementary to at least three nucleotides in said naturally occurring nucleic acid sequence adjacent to said target site.

19. The nucleic acid of claim 1, further comprising a plurality of high affinity nucleotide analogs.

20. The nucleic acid of claim 19, wherein said plurality of analogs are disposed so that no stretch of more than four consecutive naturally occurring nucleotides is present.

21. The nucleic acid of claim 1, wherein said high affinity nucleic analog is disposed at the 3′ or 5′ end.

22. The nucleic acid of claim 1, wherein said analogs are not disposed in regions capable of forming auto-dimers or intramolecular complexes.

23. (canceled)

24. A method of inhibiting the binding of a siRNA to a target site, said method comprising contacting one or more nucleic acid(s) of claim 1 with a cell expressing said siRNA and said target site.

25. The method of claim 24, wherein said contacting occurs in vitro.

26. (canceled)

27. A method of determining an off-target effect induced by a siRNA on an eukaryotic cell expressing a target site for said siRNA, comprising determining a phenotype of said eukaryotic cell after subjecting said cell to said siRNA and one or more nucleic acid(s) of claim 1, binding to a region comprising a portion of said target site.

28. The method of claim 27, wherein said determining the phenotype comprises determining in said cell the expression levels of a plurality of different genes and/or their translation products.

29. The method of claim 28, wherein said plurality of different genes comprises at least 5 different genes.

30. The method of claim 27, wherein the expression levels of a plurality of different genes is determined by array analysis.

31. A method of determining whether a phenotype induced by a siRNA in an eukaryotic cell expressing a target site for said siRNA is associated with an off-target effect, said method comprising:
a) determining the phenotype of the eukaryotic cell,
b) introducing into the eukaryotic cell said siRNA, and one or more nucleic acid(s) of claim 1, binding to a region comprising a portion of said target site,
c) determining the phenotype in the eukaryotic cell from step b., and
d) comparing the phenotype determined in step a) with the phenotype determined in step c),
wherein a difference in the phenotype determined in step a) from the phenotype determined in step c), indicates that the phenotype induced by said siRNA is associated with an off-target effect.

32. The method of claim 31, wherein step b) further comprises determining the phenotype of the eukaryotic cell after introduction of said siRNA but prior to introduction of said one or more nucleic acid(s) of claim 1.

33. The method of claim 32, wherein said determining the phenotype of the eukaryotic cell comprises determining in the eukaryotic cell the expression levels of a plurality of different genes and/or their translation products.

34. The method of claim 33, wherein said plurality of different genes comprises at least 5 different genes.

35. The method of claim 31, wherein the expression levels of a plurality of different genes is determined by array analysis.
